(19) United States
(12) Patent Application Publication
(10) Pub. No.: US 2015/0160184 A1
(43) Pub. Date: Jun. 11, 2015

(54) METHOD OF EASILY IDENTIFYING LUBRICATING OILS, IDENTIFICATION KIT AND LUBRICATING OILS THAT CAN BE EASILY IDENTIFIED

(71) Applicant: SHELL INTERNATIONAL RESEARCH MAATSCHAPPIJ B.V., The Hague (NL)

(72) Inventors: Kiyoshi Hanyuda, Kanagawa (JP); Kouichi Kubo, Kanagawa (JP); Hiroyuki Tazaki, Kanagawa (JP)

(21) Appl. No.: 14/349,682

(22) PCT Filed: Oct. 5, 2012
(86) PCT No.: PCT/EP2012/069745

§ 371 Date: Apr. 4, 2014

(30) Foreign Application Priority Data
Oct. 7, 2011 (JP) 2011-223116

(51) Int. Cl. G01N 33/28; C10M 133/06

(52) U.S. Cl. CPC G01N 33/2882; C10M 133/06

(57) ABSTRACT

Method for the identification of a lubricating oil composition, characterised in that it comprises the following steps: a heating process to heat the lubricating oil composition which is the target of the investigation and which has the possibility of containing a volatile amine as a marker; after the heating process, a first reaction process which disposes into a vapour phase on top of the liquid surface of the lubricating oil composition a medium which contains an amine colouring reagent to be used in at least one kind of amine-based colouring reaction selected from a group comprised of quinhydrone reactions, ninhydrin reactions and Dragendorff reactions; after the heating process, a second reaction process which disposes into a vapour phase on top of the liquid surface of the lubricating oil composition a medium which contains a pH-dependent colouring reagent to be used in a pH-dependent colouring reaction; and an evaluation process in which the lubricating oil composition which is the target of the investigation is assessed as to whether or not it is a specific lubricating oil composition by at least comparing a standard colouring pattern, in which a first colouring pattern attributable to the reaction or non-reaction of an amine colouring reagent and a volatile amine and a second colouring pattern attributable to the reaction or non-reaction of a pH-dependent colouring reagent and a volatile amine are displayed, with a colouring pattern which is the result of the first reaction process and the second reaction process.

### TECHNICAL FIELD

This invention relates to a method of identifying lubricating oils, a kit to be used for the identification and lubricating oils which can be thus readily identified. More specifically, it relates to a simple method for identifying genuine lubricating oils by identifying markers contained in the lubricating oils.

### BACKGROUND OF THE INVENTION

Lubricating oils are used in various kinds of mechanical apparatus in order to operate them safely and smoothly without the occurrence of seizures or wear even during long periods of use. Many kinds of lubricating oils exist and those employed are whichever match the purpose of the apparatus being used.

As required, various kinds of additives are blended with the lubricating oils in a well balanced way. For example, in the case of hydraulic apparatus for construction equipment, mainly wear inhibitors, metallic detergents and anti-oxidants are added to the base oil, whilst in the case of oils for internal combustion engines or the like, in addition to the aforementioned additives such as ashless dispersants and viscosity index improvers are added (see Tribology Handbook, Yokendo Ltd (2001/3/30 1^{st} edition), C. Lubricants p. 577-770).

To the extent that mechanical apparatus may deliver higher output, higher performance or higher durability, the lubricating oil compositions used in such cases are required to deliver similar kinds of higher performance, for example as regards anti-wear characteristics, anti-seizure characteristics and fuel economy. For this reason, lubricating oils are now high performance and extremely complex compared with the prior art, requiring a balance in the kinds, combinations and amounts of additives used (see Tribology Handbook, Yokendo Ltd (2001/3/30 1^{st} edition), C. Lubricants p. 577-770.

Accordingly, depending on the makers of the various kinds of machinery and apparatus, each company sells products for which it has approved the performance and life as its genuine (authentic) lubricating oil products, they recommend users to use these products, and they scrupulously carry out servicing for customers, paying heed to maintenance of the various kinds of apparatus. At the same time, lubricating oil manufacturers may also get their own approvals to sell their brands of lubricating oil as recommended by the makers of the machinery and apparatus. Particularly in the case of lubricating oils that can be purchased commercially, these may include products that are insufficient for performance or which are inferior, and so it is advisable to carry out oil or grease changes by procuring lubricating oils and greases which, if they exist, are the company's own authentic oils or are recommended by lubricating oil manufacturers.

However, among the lubricating oils that can be procured commercially, there exist generic products where the indications are similar to the machinery and apparatus maker's own authentic product or the branded products of the lubricating oil manufacturers, or inferior products which are sold with a similar container but filled with a different oil. If such inferior products are used unwittingly, unexpected damage to the apparatus may occur, or the due performance of the machinery may not be displayed.

Methods of identifying genuine lubricating oils have therefore been proposed in the prior art (Japanese Patent 2006-501344, Japanese Patent 2006-517998). These methods do not make the identification on the spot where the lubricating oil is actually being changed, and involve sampling oils after they have been added to the machine, sending the sample to a test centre where the test and analysis equipment has been installed, or to some other place with the appropriate research facilities, and doing the analysis and investigation there.

Although the accuracy of the results in the lubricating oil identification methods of the prior art has been high, the problem is that several days elapse before the results are available and in that period machinery is being run in a condition where it is not known whether the lubricating oil is a good product or an inferior one.

So as to be able to pre-empt problems with mechanical apparatus due to use of inferior products, this invention therefore has the aim of offering a means of identifying easily, in a short time, and at the site where the machine is actually used whether the lubricating oil is a genuine product and will have the performance indicated.

### SUMMARY OF THE INVENTION

According to the present invention there is provided a method for the identification of a lubricating oil composition, characterised in that comprises the following steps:

a heating process to heat the lubricating oil composition which is the target of the investigation and which has the possibility of containing a volatile amine as a marker;

after the heating process, a first reaction process which disposes into a vapour phase on top of the liquid surface of the lubricating oil composition a medium which contains an amine colouring reagent to be used in at least one kind of amine-based colouring reaction selected from a group comprised of quinhydrone reactions, ninhydrin reactions and Dragendorff reactions;

after the heating process, a second reaction process which disposes into a vapour phase on top of the liquid surface of the lubricating oil composition a medium which contains a pH-dependent colouring reagent to be used in a pH-dependent colouring reaction; and

an evaluation process in which the lubricating oil composition which is the target of the investigation is assessed as to whether or not it is a specific lubricating oil composition by at least comparing a standard colouring pattern, in which a first colouring pattern attributable to the reaction or non-reaction of an amine colouring reagent and a volatile amine and a second colouring pattern attributable to the reaction or non-reaction of a pH-dependent colouring reagent and a volatile amine are displayed, with a colouring pattern which is the result of the first reaction process and the second reaction process.

According to this invention, this has the effect that it is possible to offer a means of identifying easily, in a short time, and at the site where the machine is actually used whether the lubricating oil is a recommended or genuine product and will have the performance indicated.

In specific terms, since generic products or inferior products will not contain such a volatilising amine (for example, an amine-based volatile corrosion inhibitor), it is possible to use this volatilising amine (for example, an amine-based volatile corrosion inhibitor) as a marker for a recommended product or a genuine product. As a result, it is possible to prevent in good time mistaken use by a consumer of a generic product or an inferior product causing problems with machinery. In addition, given that the volatilising amine which is the marker (for example, an amine-based corrosion inhibitor) is volatile, easy detection of the marker is possible by means of a simple method at room temperature or after heating the material being investigated (for example, to about 60 to 70° C.) using hot water in a pot on site.

### BRIEF EXPLANATION OF DRAWINGS

FIG. 1 is a schematic drawing of a first embodiment of the method of identification of the present invention. Specifically, it is a test for identifying a lubricating product by means of a colouring reaction (for example, in the case where a pH reagent paper and a quinhydrone reagent paper are used).

FIG. 2 is a schematic drawing of a second embodiment of the method of identification of the present invention. Specifically, it is a test for identifying a lubricating product by means of a colouring reaction (in the case where a gas detection tube is used).

### DETAILED DESCRIPTION OF THE INVENTION

This invention is a method for the identification of a lubricating oil composition, characterised in that it comprises the following steps:

a heating process to heat the lubricating oil composition which is the target of the investigation and which has the possibility of containing a volatile amine as a marker;

after the heating process, a first reaction process which disposes into a vapour phase on top of the liquid surface of the lubricating oil composition a medium which contains an amine colouring reagent to be used in at least one kind of amine-based colouring reaction selected from a group comprised of quinhydrone reactions, ninhydrin reactions and Dragendorff reactions;

after the heating process, a second reaction process which disposes into a vapour phase on top of the liquid surface of the lubricating oil composition a medium which contains a pH-dependent colouring reagent to be used in a pH-dependent colouring reaction; and

an evaluation process in which the lubricating oil composition which is the target of the investigation is assessed as to whether or not it is a specific lubricating oil composition by at least comparing a standard colouring pattern, in which a first colouring pattern attributable to the reaction or non-reaction of an amine colouring reagent and a volatile amine and a second colouring pattern attributable to the reaction or non-reaction of a pH-dependent colouring reagent and a volatile amine are displayed, with a colouring pattern which is the result of the first reaction process and the second reaction process.

Given below is an explanation of a lubricating oil composition which is the target of the investigation and which is a recommended or genuine product, after which an explanation of the method of identifying lubricating oil compositions which pertains to this invention is given, and then an explanation of the kit for identifying lubricating oil compositions which pertains to this invention is given.

A lubricating oil composition which is the target of the investigation and which is a recommended or genuine product is a lubricating oil composition in which at least one kind of volatile amine has been added to a base oil as a marker and which may also contain other additives as required. Each constituent is described below.

The base oil in the lubricating compositions of this invention denotes one of those generally capable of being used as base oils for lubricating oils, and mention may be made of synthetic oils, mineral oils and base oils obtained from GTL processes, for example those of Groups I to V. Groups I, II, III, IV and V here are the broad classifications of base oil blending components defined by the American Petroleum Institute prepared as a guide to lubricating oil base oils. The suitable base oils can be varied according to the application, and selected easily and reliably by a manufacturer.

The volatile amines in the lubricating oil compositions of this invention are not specially limited provided they are volatilising amines. Suitable ones are tertiary amines, quaternary ammonium salts, and also any aliphatic amines and aromatic amines, and they may also be amine salts. Among amines, primary amines have the strongest basicity and because the reactivity of the hydrogen atoms which are bonded with the nitrogen atoms is also high, there is a possibility that they will have a major effect as regards characteristics and performance on the product to which they are added, which is not desirable. Also, in the case of secondary amines, if the products to which they are added contain nitrous acid compounds (it is possible in particular that engine oils may contain nitrous acid gases in the blow-by gases), nitrosoamines which are potentially carcinogenic may occur, which is not desirable. Here, what is meant by “volatile” is becoming vaporised at not more than 100° C. (the lower-bound temperature is not specially limited, and may for example be 0° C.) As a more specific example, mention may be made of amines where vaporisation is promoted when they are heated by taking advantage of hot water or the like in a pot on site when an investigation of genuineness of a product is being made. The reason why vaporisation at not more than 100° C. is ideal is that normal additives added to lubricating oils do no vaporise at below 100° C. In other words, it is because (1) when investigating whether a product is a recommended one or a genuine one, the normal additives are not effected by vaporisation, and (2) the normal additives do not end up escaping from the lubricating oil by vaporising, so that it is possible to prevent problems whereby the performance required of said normal additives cannot be adequately displayed. A more preferable vaporisation temperature here is 40 to 90° C. The reason why above 40° C. is preferred is so that, when a container cap is left off for a long period (in an open system) while fresh oil is being drawn off from the container (a drum or the like), the amine concentration in the fresh oil does not drop through vaporisation of the volatile amines before the identification investigation is done (similarly also in the case of making a confirmation after a recommended oil containing amines is loaded into a machine's tank). Also, the reason why up to 90° C. is preferred is that, in the case of confirmation by boiling hot water or the like when confirming on site, it may be supposed that the temperature of the hot water will fall by the time confirmation is made. 50 to 80° C. is even more preferred. A lubricating oil is used in industry at 40 to 60° C. and in cars at 80 to 100° C.

From the standpoint of being acceptable as a volatile amine to the extent it has no impact on performance of the lubricating oil and has merit as a corrosion inhibitor, a volatile amine-based corrosion inhibitor [also called a VCI (volatile corrosion inhibitor)] is preferred here. A volatile amine-based corrosion inhibitor is a compound or a mixture of several such which vaporises slowly at room temperature. The vaporised amine is chemically or physically adsorbed onto or reacts with the surface of a metal, as a result of which corrosion of the metal is inhibited or prevented (cited from page 16 of Recent Trends in Corrosion Inhibition Prevention Technology, Junkatsuyu Keizai (Lubricating Oil Economics), No. 524 (2009) p. 1-31). The volatile amine-based corrosion inhibitor is vaporised simply at room temperature or by a slight degree of heating, so that it can be detected easily even without immersing the test paper used for detection into the lubricating oil. Examples of amine-based volatile corrosion inhibitors that are ideal for use are trialkylamines (alkyldiisopropylamines, alkyldiisobutylamines, alkyldioctylamines, tributylamines and the like), cycloalkylamines (alkyldicyclohexylamines, dicyclohexylamine and the like), diethylaniline, dialkylanilines, dialkenylanilines, dibenzylamine, tribenzylamine and alkanol amines (triethanolamines and the like). Amine-based volatile corrosion inhibitors may also be salts of acids (for example, benzoic acid, cyclohexanecarboxylic acids, nitrous acid, hydrochloric acid, acrylic acid and salicylic acid). Of these, tertiary amines are preferred, and tributylamines especially preferred, for the following reasons. First, even when a machine is being exported or apparatus is being shut down for a long period, said constituent vaporises from the oil and is adsorbed onto the metal surfaces of parts that are not immersed in oil, and there is a volatile corrosion inhibiting effect which prevents rusting of the metal surface due to moisture in the air. Furthermore, even within the oil these amines compounds try to adsorb onto metal surfaces because of polarisation due to the unpaired electron pairs of the nitrogen atoms, but in the case of tertiary amines such as tributylamines hydrocarbon groups bond on the periphery of the nitrogen atoms, so that because of steric hindrance they do not adsorb very strongly onto metal surfaces compared with other anti-wear agents and oiliness agents, and they are unlikely to impact on the anti-friction properties of the lubricating oil being used. In addition, in hydraulic oils, for example, zinc dialkyldithiophosphates (ZnDTP) are added as anti-wear agents. In such systems, even if a tertiary amine in which three hydrocarbon groups bond to the nitrogen atom is used as a marker, the unpaired electron pair portion of the nitrogen atom is unlikely to coordinate with the zinc metal atoms of the ZnDTP simply because of steric hindrance, and so there is no detrimental impact on the anti-friction properties of the ZnDTP.

The amount of volatile amine (amine-based volatile corrosion inhibitor) is preferably an amount that has no impact on performance of the lubricating oil (for example, on anti-friction performance). Said amount depends on the constituents and composition of the lubricating oil to which it is added, the application of the lubricating oil, and the kind of volatile amine (amine-based volatile corrosion inhibitor) selected. Normally, the amount of amine-based volatile corrosion inhibitor, relative to 100 mass % of lubricating oil base oil composition, is preferably 0.05 to 2.0 mass %, but more preferably 0.06 to 1.75 mass % and even more preferably 0.07 to 1.5 mass %.

As mentioned above, when adding a volatile amine (amine-based volatile corrosion inhibitor) to a lubricating oil composition, as well as adding to the lubricating oil product a volatile amine (amine-based volatile corrosion inhibitor) which is a candidate for use as a marker, it is necessary beforehand, in regard to which constituents to select and how much of an amount is to be added, to determine the amount of volatile amine to be added by verifying whether or not there is any impact on the properties and performance of said lubricating oil product. It is then possible for a manufacturer to determine easily how much of what sort of marker is to be added.

For example, not significantly altering the characteristics and performance of the lubricating oil to which the volatile amine is added, such as aroma of the product, flash point, density, anti-wear properties and so on, can be a factor in the kind of volatile amine and the amount added. Taking account also of the environmental aspects round about, it is preferable not only not to alter the characteristics and performance of the product but also to keep the amount added as low as possible. Given these points, as mentioned above the amount added may generally be considered to be in the range 0.05 to 2.0 mass %. In particular, if the view is taken that the amount added ought to be to the extent that the flash point of the product to be used will not be lowered, then not more than 1 mass % is good, and not more than 0.2 mass % is even better.

Apart from the above mentioned volatile amine (amine-based volatile corrosion inhibitor), it is possible to use as required one kind or more of optional additives in the lubricating oil compositions of this invention, such as anti-wear agents, metal deactivators, anti-static agents, defoamers, anti-oxidants, dispersants, detergents, extreme pressure agents, friction modifiers, viscosity index improvers, pour point depressants, tackifiers, metallic detergents, ashless dispersants and corrosion inhibitors. It is possible, for example, to use “additives packages” (for example, various kinds of ATF additives packages) as used for improving performance.

This method of the present invention is characterised in that it has the following steps:

a heating process to heat the lubricating oil composition which is the target of an investigation and which has the possibility of containing a volatile amine as a marker; after the heating process, a first reaction process which disposes into a vapour phase on top of the liquid surface of the lubricating oil composition a medium which contains an amine colouring reagent to be used in at least one kind of amine-based colouring reaction selected from a group comprised of quinhydrone reactions, ninhydrin reactions and Dragendorff reactions; after the heating process, a second reaction process which disposes into a vapour phase on top of the liquid surface of the lubricating oil composition a medium which contains a pH-dependent colouring reagent to be used in a pH-dependent colouring reaction; and an evaluation process in which the lubricating oil composition which is the target of the investigation is assessed as to whether or not it is a specific lubricating oil composition by at least comparing a standard colouring pattern, in which a first colouring pattern attributable to the reaction or non-reaction of an amine colouring reagent and a volatile amine and a second colouring pattern attributable to the reaction or non-reaction of a pH-dependent colouring reagent and a volatile amine are displayed, with a colouring pattern which is the result of the first reaction process and the second reaction process.

The heating process pertaining to this invention is a process for heating the lubricating oil composition which is the target of the investigation and which has the possibility of containing a volatile amine as a marker {for example, up to a temperature of at least approximately 70° C. (more preferably at least approximately 90° C.)}. The method of heating and heating conditions here are not specially limited so long as the conditions are such that the volatile amine will vaporise to the extent where measurement is possible. For example, from the standpoint of comparative ease of investigation procedures on site, mention may be made of a method of heating the sample to be investigated to 60 to 70° C. by using hot water in a pot.

The first reaction process pertaining to this invention is a process which, after a heating process, disposes into a vapour phase on top of the liquid surface of the lubricating oil composition a medium (for example, quinhydrone test paper impregnated with quinhydrone) which contains an amine colouring reagent to be used in at least one kind of amine-based colouring reaction selected from a group consisting of quinhydrone reactions, ninhydrin reactions and Dragendorff reactions (for example, said medium is suspended in the reaction vessel). By being subjected to this process, the amine colouring reagent will (or will not) be coloured by virtue of any amine which is vaporised by the heating process. For example, in the case of an amine colouring reagent based on the quinhydrone reaction (quinhydrone), a primary amine produces the colour purple, a secondary amine red, a tertiary amine yellowy orange, and a quaternary amine greenish yellow. It is also possible to combine a plurality of amine colouring reagents. Also, it is possible to determine easily according to the manufacturer which amine colouring reagents are to be used on the basis of the kinds of volatile amines targeted by the reaction.

The medium containing the amine-based colouring reagent here is not specially limited, but from the standpoint of comparative ease of investigation procedures on site, it is ideal if it is a reagent paper. For example, said reagent paper can be obtained by suffusing the amine-based colouring reagent onto filter paper and then evaporating off the solvent.

The second reaction process pertaining to this invention is a process which, after a heating process, disposes into a vapour phase on top of the liquid surface of the lubricating oil composition a medium (for example, a universal pH test paper in which filter paper is impregnated with thymol blue and cresol red) which contains a pH-dependent colouring reagent to be used in pH-dependent colouring reactions (for example, said medium is suspended in the reaction vessel). By being subjected to this process, the pH-dependent colouring reagent will (or will not) be coloured by virtue of any amine which is vaporised by the heating process. It is also possible to combine a plurality of pH-dependent colouring reagents. Also, it is possible to determine easily according to the manufacturer which pH-dependent colouring reagents are to be used on the basis of the kinds of volatile amines targeted by the reaction. It is possible to carry out the first and second reaction processes here simultaneously, or it is possible to carry out the first reaction process first and then the second reaction process, or to carry out the second reaction process first and then the first process.

The medium containing the pH-dependent colouring reagent here is not specially limited, but from the standpoint of comparative ease of investigation procedures on site, it is ideal if it is a reagent paper. For example, said reagent paper can be obtained by suffusing the pH-dependent colouring reagent onto filter paper and then evaporating off the solvent.

The reason for using the first reaction process and the second reaction process together is to increase the accuracy of the amine detection. In other words, with one kind of colouring reaction process, if makers have by chance added markers to lubricating oils which present the same colours, it is absolutely impossible to assess the authenticity of the lubricating oil, but when two or more kinds of reaction processes are implemented, the likelihood of the same colouring reactions being obtained by chance is reduced and so the authenticity of the lubricating oil composition targeted for identification can be assessed more accurately. The reaction processes are not limited to the two first and second reaction processes. To increase accuracy, further reaction processes may also be added.

The evaluation process is a process in which a lubricating oil composition which is the target of an investigation is assessed as to whether or not it is a specific lubricating oil composition by at least comparing a standard colouring pattern, in which a first colouring pattern attributable to the reaction or non-reaction of an amine colouring reagent and a volatile amine and a second colouring pattern attributable to the reaction or non-reaction of a pH-dependent colouring reagent and a volatile amine are displayed, with a colouring pattern which is the result of the first reaction process and the second reaction process. As a result, it is possible to identify a lubricating oil composition as genuine and to distinguish it from other kinds of oil.

The investigation pertaining to this invention may be carried out at any time. For example, it may be timed for the actual change of lubricating oil composition at the site where it is used, or it may be timed to be immediately before filling the equipment with it.

The standard colouring pattern is based on the kind of volatile amine added (amine-based volatile corrosion inhibitor), the kind of amine-based colouring reagent used, and the conditions established for the reaction (such as time and temperature). It is necessary to confirm how changes in colour due to reagents are obtained by the chemical colouring reaction. The standard colouring pattern is preferably in the form of a standard colouring sheet.

The identification kit pertaining to this invention comprises:

a medium (for example, reaction test paper) which contains a reaction reagent to be used in at least one kind of amine-based colouring reaction selected from the group consisting of quinhydrone reactions, ninhydrin reactions and Dragendorff reactions,

a medium (for example, pH test paper) which contains a reaction reagent to be used in a pH-dependent colouring reaction, and

a standard colouring pattern, in which a first colouring pattern attributable to the reaction or non-reaction of an amine colouring reagent and a volatile amine and a second colouring pattern attributable to the reaction or non-reaction of a pH-dependent colouring reagent and a volatile amine are displayed. The standard colouring pattern does not necessarily have to be in the kit and may be held by the person doing the measurements at the time of measurement.

The medium (for example, reaction test paper) which contains the reaction reagent to be used in the amine-based colouring reaction and the medium (for example, pH test paper) which contains the reaction reagent to be used in the pH-dependent colouring reaction are preferably in separate forms, but it is also possible to use them in an integrated form (for example, where the reaction reagent to be used in the amine-based colouring reaction is incorporated at a specific point on the filter paper, and the reaction reagent to be used in the pH-dependent colouring reaction is incorporated at a different specific point on said filter paper). Furthermore, the media (or medium if only one is used instead of two) containing these reaction reagents can also be integrated with a standard colouring sheet {for example, the reaction reagent to be used in the amine-based colouring reaction is incorporated at a specific point on the filter paper, and close to it is disposed standard colouring which shows the colouring pattern when the amine-based colouring reagent reacts (or does not react) with the volatile amine, and the reaction reagent to be used in the pH-dependent colouring reaction is incorporated at a different specific point on the filter paper, and close to it is disposed standard colouring which shows the colouring pattern when the pH-dependent colouring reagent reacts (or does not react) with the volatile amine).

Below is given an explanation of concrete procedures to detect whether or not an amine-based volatile corrosion inhibitor is present in a lubricating oil composition product, in other words whether or not it is a recommended or genuine product. However, this is only one example and the technical scope of the invention is in no way limited to this example.

Procedure 1: Before applying the lubricating oil to the machinery, a specified amount (for example, approximately 100 cc) is taken as a sample into a sealable container (for example, a triangular flask) of specific capacity (for example, 200 cc) from a container (for example, a drum, a 20-litre pail, or a 4-litre tank) which has been filled with said lubricating oil.

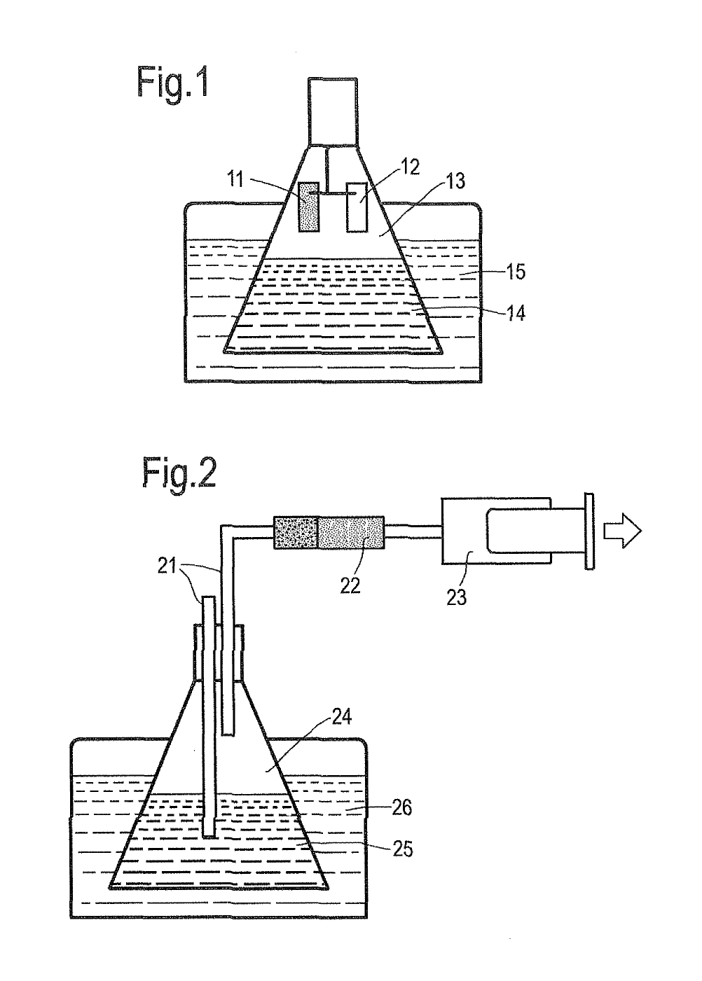

Procedure 2: As shown in FIG. 1, in the upper part of the sealable container (for example, a triangular flask) are placed a medium 11 containing a pH-dependent colouring reagent (for example, universal pH test paper) and a medium 12 containing an amine colouring reagent (for example, quinhydrone test paper), and the container is sealed. The container with the test specimen inside (for example, a triangular flask) is placed in a hot water bath for a specified time (for example, approximately 10 minutes), the sampled test specimen thus being heated.

Procedure 3: If it is a lubricating oil product identifiable by the addition of an amine-based volatile corrosion inhibitor as a marker, the volatile corrosion inhibitor, through being heated, fills the inside of the container (for example, a triangular flask) and, assuming for example universal pH test paper impregnated with thymol blue and cresol red where blue shows alkali and for example quinhydrone test paper impregnated with quinhydrone reagent, a chemical colouring reaction according to the amine compound is obtained (in general the colours are purple for primary amines, red for secondary amines, yellowy orange for tertiary amines and, depending on the case, greenish yellow for quaternary amines).

Procedure 4: A comparison is made between the standard colouring pattern and the colour of the result obtained by heating at the actual site, and if the same result is obtained, it may be deemed that the test specimen sampled on site is a recommended oil or genuine product identified by the volatile amine used as a marker.

### Examples of Other Applications

### (1) Kinds of Markers

In this invention a volatile amine is selected as a marker in order to identify a lubricating oil easily. This is because it has been observed that it is possible to identify whether a lubricating oil is genuine or not on the basis of reactions of the functional groups known as amine groups (primary, secondary, tertiary and quaternary), and thus on the basis of their colouring reactions. However, apart from offering significant results, it does not have a detrimental impact on the performance of the lubricating oil, and based also on ease of procurement and cost tertiary and quaternary volatile amines alone have been selected on the assumption of being for practical use, but in theory other kinds of markers can also be used. It is also possible to use volatile amines as illustrated in this invention and other kinds of markers in combination.

For example, for the constituent having the function of the volatile corrosion inhibitor in one example of this invention, it is possible to use aliphatic esters, salts of amines and aliphatics, and heterocyclic compounds such as triazoles and thiazoles. Suitable for use, for example, are volatile corrosion inhibitors such as benzotriazole, tolyltriazole, dicyclohexyl ammonium nitrite, monoethanolamine benzoate, dicyclohexyl ammonium benzoate, diisopropyl ammonium benzoate, diisopropyl ammonium nitrite, cyclohexylamine benzoate, cyclohexylammonium, cyclohexanecarboxylates, dicyclohexyl ammonium acrylate and dicyclohexyl ammonium salicylate (Recent Trends in Corrosion Inhibition Prevention Technology, Junkatsuyu Keizai (Lubricating Oil Economics), No. 524 (2009) p. 1-31). In the case also of selecting constituents other than volatile amines, if adding them as a marker it is ideal to incorporate them in an additive concentration within a range that does not have a detrimental impact on the characteristics and performance of the lubricating oil product which it is desired to identify.

### (2) Functions Possessed by Marker

In this invention, the marker in one example has been assumed to have a corrosion inhibiting function. The marker is, as mentioned above, present in very small amounts in the lubricating oil composition. However, even if present in very small amounts, it is necessary to prevent it to the utmost having an essentially detrimental effect on the lubricating oil composition. From this standpoint, if the marker is to be present during actual use, ideally there should be essentially no detrimental impact on the lubricating oil composition, and rather it should be a constituent which contributes good properties to the lubricating oil composition. Therefore, from the standpoint of effecting prevention of corrosion of machinery during use, it is appropriate to have, as shown in the example, tertiary and quaternary amine-based volatile corrosion inhibitors as the volatile amine. However, proceeding from the basic aim of this invention, there is no restriction on the constituent which has the corrosion-inhibiting properties, provided that (i) even if the marker is present during use it is a constituent which essentially has no detrimental impact on the lubricating oil's performance it may basically be any kind of constituent, and further provided that if (ii) it is a constituent where there is a risk that the marker being present during use will have a detrimental impact on the lubricating oil's performance, it is necessary to make the amount of marker present in the lubricating oil composition an amount such that there is essentially no impact on the lubricating oil's performance.

### (3) Method of Detecting Marker

In this invention, from the standpoint of being able to carry out detection procedures with comparative ease on site, colouring reactions have been presented as an ideal example of a method of detection. However, the method of detection is not specially limited and, as shown for example in FIG. 2, it is possible to carry out the procedure in a more straightforward and easier way also in a method of detection using a gas detection type of method in which a glass tube is packed with silica gel onto which an indicator has been adsorbed. More specifically, by using two or more kinds of gas detection tubes which detect a marker, it is also possible to detect and/or confirm in similar fashion (for example, Kitakawa gas detection tubes (made by Komyo Rikagaku Kogyo Ltd) and Gastec detection tubes (made by Gastec Co. Ltd)). These gas detection tubes naturally detect amines (for example, tributylamines) but can also detect ammonia, sulphides (constituents containing S atoms) and, for example, thiazoles and triazoles.

### Examples

The invention is further explained in concrete form by way of examples and a comparative example below, but the invention is not limited by the following.

In order to show that application was possible also to various kinds of lubricating oil products, one litre of a process-A oil in which the composition comprised 100 mass % API Group III base oil was used and to this tributylamine was added as an amine-based volatile corrosion inhibitor so as to comprise 0.1 to 1.0 mass %. It was dissolved by agitating the lubricating oil containing the amine-based volatile corrosion inhibitor for 30 minutes at 50° C. One-litre steel sample containers (5×11×18 cm) were filled with the mixtures and the cap was tightly sealed. They were left to stand for a full day and night at room temperature (20° C.) 100 ml samples were drawn off from the steel sample containers containing each test specimen. Using a 200 ml triangular flask, quinhydrone reagent paper and universal pH reagent paper were placed inside the triangular flask in the arrangement as shown in FIG. 1. The triangular flask with the reagent papers disposed within was heated with hot water by adding hot water kept at about 90° C. inside a thermos flask to a one-litre beaker. The state of the reagent papers was verified after about 10 minutes. The results are shown in Table 1.

It was confirmed that there was a colouring reaction by which it was possible to make a satisfactory identification even with a sample of process-A oil to which 0.1 mass % of tributylamine, which is an amine-based volatile corrosion inhibitor, had been added.

TABLE 1

| | | | | | Comparative |
|---|---|---|---|---|---|
| | | Example 1 | Example 2 | Example 3 | Example 1 |
| Composition | Process-A oil | | | | |
| | Lubricating oil | 99.9 | 99.8 | 99 | 100 |
| | base oil, mass % | | | | |
| | Tributylamine, mass % | 0.1 | 0.2 | 1 | — |
| Evaluation | All-purpose ‘UNIV’ | Colouring | Colouring | Colouring | Colouring |
| | universal pH test paper, | reaction: | reaction: | reaction: | reaction: |
| | made by Advantec Group | Yes - blue | Yes - blue | Yes - blue | No - no change |
| | Quinhydrone test | Colouring | Colouring | Colouring | Colouring |
| | paper (Note 1) | reaction: | reaction: | reaction: | reaction: |
| | | Yes - pink | Yes - pink | Yes - pink | No - no change |
| | Kinematic viscosity | 19.4 | 19.4 | 19.4 | 19.4 |
| | @40° C., mm²/s | | | | |
| | Density (15/4° C.), g/cm³ | 0.834 | 0.834 | 0.834 | 0.834 |
| | Pour point, ° C. | −15 | −15 | −15 | −15 |
| | Flash point (COC), ° C. | 230 | 230 | 228 | 230 |
| Remarks | Remarks | Same Charac- | Same charac- | Same charac- | |
| | | teristics as | teristics as | teristics as | |
| | | Process-A oil | Process-A oil | Process-A oil | |

(Note 1):
The quinhydrone test paper was made as follows. A 3.0 mass % solution of quinhydrone was prepared by using a 50 mass % aqueous solution of ethanol as solvent. The ethanol aqueous solution with the 3.0 mass % quinhydrone was suffused onto a normal filter paper (Qualitative Filter Paper No. 1 made by Advantec Group). When the filter paper had absorbed sufficient solution, it was made ready for use by drying it for about 10 minutes at room temperature (20° C.).

From the results of Table 1 it can be seen that identification is possible by the method of this invention. Furthermore, no special analytical apparatus was necessary. The identification could be made easily and on the site where the machinery was actually used provided test papers (or detection tubes) capable of detecting the compound in the volatile corrosion inhibitor and small test sample containers were provided in advance.

### EXPLANATION OF SYMBOLS

11 Colour change due to universal pH test paper
12 Colour change due to quinhydrone test paper
13 Vaporised amine-based corrosion inhibitor
14 Sampled test material (product to be identified)
15 Hot-water bath for heating
21 Glass tube
22 Glass detection tube
23 Cylinder for suction
24 Vaporised amine-based corrosion inhibitor
25 Sampled test material (product to be identified)
26 Hot-water bath for heating.

1. A method for determining the presence or absence of a volatile amine as a marker in a lubricating oil composition comprising;
heating the lubricating oil composition which has the possibility of containing the volatile amine;
after the heating, disposing a medium comprising an amine colouring reagent into a vapour phase on top of the liquid surface of the lubricating oil composition to obtain a first colouring pattern, wherein the amine colouring reagent will react with the volatile amine, if present, in an amine-based colouring reaction selected from the group consisting of quinhydrone reactions, ninhydrin reactions and Dragendorff reactions, and wherein the first colouring pattern is attributable to the occurrence or non-occurrence of the amine-based colouring reaction;
after heating, disposing a medium comprising a pH-dependent colouring reagent into a vapour phase on top of the liquid surface of the lubricating oil composition to obtain a second colouring pattern, wherein the pH-dependent colouring reagent will react with the volatile amine, if present, in a pH-dependent colouring reaction, and wherein the second colouring pattern is attributable to the occurrence or non-occurrence of the pH-dependent colouring reaction; and
determining the presence or absence of the volatile amine in the lubricating oil composition by comparing the first colouring pattern and the second colouring pattern with a standard colouring pattern.

2. The method of claim 1 wherein the volatile amine is a tertiary amine which vaporises at not more than 100° C.

3. The method of claim 1 wherein the volatile amine is at least one kind selected from a group consisting of tributylamines, dimethylethylamines and triethylamines.

4. The method of claim 1 wherein the medium comprising the amine colouring reagent and/or the medium comprising the pH-dependent colouring reagent.

5. A kit for determining the presence or absence of a volatile amine as a marker in a lubricating oil composition comprising:
a medium that comprises an amine colouring reagent, wherein the amine colouring reagent is capable of reacting with the volatile amine in an amine-based colouring reaction to produce a first colouring pattern, and wherein the amine-based colouring reaction is selected from the group consisting of quinhydrone reactions, ninhydrin reactions and Dragendorff reactions; and
a medium that comprises a pH-dependent colouring reagent, wherein the pH-dependent colouring reagent is capable of reacting with the volatile amine in a pH-dependent colouring reaction to produce a second colouring pattern.

6. A lubricating oil composition comprising a base oil and a volatile amine as a marker, and wherein it is possible to identify the lubricating oil composition by means of the method of claim 1.

7. The lubricating oil composition of claim 6 wherein the volatile amine is a tertiary amine which vaporises at not more than 100° C.

8. The lubricating oil composition of claim 6 wherein the volatile amine is at least one kind selected from the group consisting of tributylamines, dimethylethylamines and triethylamines.

9. The kit of claim 5 further comprising a standard colouring pattern comprising:
a colouring pattern attributable to the occurrence of an amine-based colouring reaction between the amine colouring reagent and the volatile amine, and
a colouring pattern attributable to the occurrence of a pH-dependent colouring reaction between the pH-dependent colouring reagent and the volatile amine.

10. The kit of claim 5 wherein the medium that comprises the amine colouring reagent and/or the medium that comprises the pH-dependent colouring reagent are reagent papers.

11. The method of claim 1 wherein the lubricating oil composition is heated to a temperature sufficient to vaporize the volatile amine.

12. The method of claim 1 wherein the lubricating oil composition is heated to a temperature of not more than 100° C.

13. The method of claim 12 wherein the volatile amine is a tertiary amine.

14. The method of claim 1 wherein the lubricating oil composition is heated to a temperature of from 40 to 90° C.

15. The method of claim 1 wherein the lubricating oil composition is heated to a temperature of from 50 to 80° C.

16. The method of claim 1 wherein the amine colouring reagent is quinhydrone.

17. A method for determining the identity of a lubricating oil composition comprising:
obtaining a sample of the lubricating oil composition;
heating the sample of the lubricating oil composition to a temperature of not more than 100° C.;
after heating, disposing a medium comprising an amine colouring reagent into a vapour phase on top of the liquid surface of the lubricating oil composition to obtain a first colouring pattern, wherein the amine colouring reagent will react with a volatile amine, if present, in an amine-based colouring reaction selected from the group consisting of quinhydrone reactions, ninhydrin reactions and Dragendorff reactions, and wherein the first colouring pattern is attributable to the occurrence or non-occurrence of the amine-based colouring reaction;
after heating, disposing a medium comprising a pH-dependent colouring reagent into a vapour phase on top of the liquid surface of the lubricating oil composition to obtain a second colouring pattern, wherein the pH-dependent colouring reagent will react with a volatile amine, if present, in a pH-dependent colouring reaction, and wherein the second colouring pattern is attributable to the occurrence or non-occurrence of the pH-dependent colouring reaction; and
determining the identity of the lubricating oil composition by visually comparing the first colouring pattern and the second colouring pattern with a standard colouring pattern.

18. The method of claim 17 wherein the volatile amine is at least one kind selected from a group consisting of tributylamines, dimethylethylamines and triethylamines.
